(19) United States
(12) Patent Application Publication
(10) Pub. No.: US 2020/0276379 A1
(43) Pub. Date: Sep. 3, 2020

(54) TISSUE CUFF

(71) Applicant: BioSurfaces, Inc., Ashland, MA (US)

(72) Inventors: Matthew D. Phaneuf, Ashland, MA (US); Dirk Hentschel, Cambridge, MA (US); John Lucas, III, Greenwood, MS (US)

(21) Appl. No.: 16/803,494

(22) Filed: Feb. 27, 2020

Related U.S. Application Data
(60) Provisional application No. 62/811,971, filed on Feb. 28, 2019.

(51) Int. Cl. A61M 1/36; A61L 27/54; D01D 5/00; D01F 6/62; D01D 1/02; D04H 1/728

(52) U.S. Cl. CPC A61M 1/3655; A61L 27/54; D01D 5/0038; D01F 6/62; D01D 1/02; B82Y 5/00; A61M 2205/04; A61M 2205/3334; A61M 2207/10; A61M 2205/02; D04H 1/728

(57) ABSTRACT

A tissue compression cuff comprises a first fastening portion defining one or more openings and at least one band. The at least one band comprises a first end extending from the first fastening portion at a first end, and an opposing second end, the at least one band defining a first diameter at the first end and a second diameter at the second end. The first diameter is larger than the second diameter. A second fastening portion is positioned at the opposing second end of the at least one band. The second end of the at least one band is configured to be inserted through the one or more openings of the first fastening portion. The tissue compression cuff is sized and configured to reduce blood flow through an arteriovenous fistula and made from an electrospun textile made up of biocompatible nanofibers.

### CROSS REFERENCE TO RELATED APPLICATION

Pursuant to relevant portions of 35 U.S.C. § 119 and 37 C.F.R. § 1.53, this application claims the benefit and priority of U.S. Patent Application 62/811,971, filed on Feb. 28, 2019, the entire contents of which is hereby incorporated by reference.

### TECHNICAL FIELD

This application is generally directed to the field of biocompatible medical implants, specifically tissue cuffs, and in one embodiment is directed to an improved fistula cuff or band that reduces blood flow through an arteriovenous fistula thereby improving hemodialysis treatment for patients with high flow access.

### BACKGROUND

Tissue cuffs are medical devices that are surgically implanted in various parts of the body to compress tissue or otherwise restrict the flow of material or the volume of a biological conduit or pouch, such as a bronchial tube, stomach, intestine, blood vessel or a synthetic and/or biologic implantable device (e.g. vascular graft). One example of a tissue cuff is a fistula cuff used in patients undergoing hemodialysis treatment. Hemodialysis is a treatment for kidney failure that uses a machine to filter the patient's blood (a dialyzer) outside the body. This procedure requires forming a vascular access, which is a site where needles are inserted to transport the patient's blood to and from the hemodialysis machine, making the life-saving treatment possible. The preferred hemodialysis access is an arteriovenous fistula (AVF), which is a connection, made by a surgeon, of an artery to a vein. Typically, the surgeon places an AVF in the forearm or upper arm. The AVF causes extra pressure and extra blood to flow into the vein. It is estimated that 2-4% of all AVFs will develop high-flow access (HFA) issues, which is defined as an access with a blood flow rate greater than 2 liters/minute. While the etiology and risk factors for developing high-flow access are unknown, ongoing arterial and venous remodeling are postulated to play a role in access dysfunction. HFA, if left untreated, can increase the incidence of cardiovascular death, hand ischemia/Steal syndrome, or promote the growth of aneurysms with potential for rupture, even though most patients appear asymptomatic.

Surgical banding, which has been used for over 30 years, is an effective HFA treatment option. Using this technique, a band made from 2-0 Prolene suture, pericardial (tissue harvested from around the heart) patch material, or synthetic vascular graft material is wrapped around the access venous tract to narrow vessel diameter followed by anchoring. The AVF outflow resistance is increased while access flow is reduced. Current banding devices or fistula cuffs suffer from issues such as lack of tissue incorporation/integration, rupture/dislocation, fibrosis, over-compliance/stretch and vessel/material mismatch, resulting in either allowing high flow to recommence or damage to the AVF. Thus, there is a need to develop an “off-the shelf” fistula cuff that would be easily implanted using current surgical techniques, can be adjusted and hold desired constrictive forces on the vein, can prevent stretching and allow surrounding tissue integration to secure the device.

A majority of medical devices such as tissue cuffs, and specifically fistula cuffs, are made from standard textile materials that have several major issues when used in medical applications. These issues include: lack of adequate device design to control constriction forces; lack of healing that stymies long term effectiveness; failure to prevent complications such as fibrosis; inflammation; and infection and an inability to provide localized therapeutic delivery of drug(s) at the disease site. Even with these issues, the materials comprising various medical devices have not undergone any major changes for over 55 years. These are just some of the shortcomings present in current tissue cuffs and specifically fistula cuffs or banding devices.

### SUMMARY

The current disclosure is directed to a novel tissue cuff or tissue compression cuff to be used to restrict the flow of material or the volume of a biological conduit or pouch such as a fistula cuff (band), using electrospinning technology (combined with standard textile technology as an option) and advanced manufacturing techniques in order to promote tissue incorporation/healing, provide strength and prevent excessive stretch and enable customization of various device dimensions, as needed. The electrospun tissue cuff permits adjusting compression of the device around the tissue or biological conduit, allowing the device to be further secured with sutures or clips.

The core technology is a process for producing nanofibrous materials out of commonly used FDA-approved polymers. These materials, which have a spider web-like composition, can form a variety of unusual and difficult-to-manufacture shapes with vastly improved healing properties as compared to other textiles and which are particularly applicable to various medical devices. This improved healing response is due to the structural similarity of the electrospun material to the structure of natural tissue in the body.

Aspects of electrospinning technology are further described in U.S. Pat. Nos. 7,413,575, 8,771,582, 10,328,032, and 10,441,550, as well as U.S. patent application Ser. No. 11/366,165 (now published as U.S. Patent Application Publication No. 2006/0200232 A1), the contents of which are hereby incorporated by reference. The electrospinning process is done at room temperature (about 25° C.) and can therefore easily incorporate drugs or other bioactive agents directly into the nanofibers of the electrospun materials used in conjunction with the fistula or other tissue cuff. This feature of the materials is important, because many existing materials must be manufactured at high temperatures. Some drugs, such as new, protein-based drugs, are sensitive to higher temperatures, meaning that it is impractical to incorporate them during the manufacturing process used for many device materials today.

A method of forming a tissue cuff from a fabricated textile comprised of electrospun nanofibers comprises dissolving a non-biodegradable polymer in an organic solvent to produce a polymer solution. The polymer solution is loaded into an electrospinning instrument configured to be set at a specified flow rate. An electrical current of 15-30 kV is applied to a needle of the electrospinning instrument. The needle is placed 5 cm-50 cm from the mandrel or collecting surface. The polymer solution is electrospun onto a mandrel for a first period of time to form an electrospun polymer material, wherein the electrospinning occurs at room temperature. In one embodiment, a polyester yarn mesh is braided onto the electrospun polymer material on the mandrel after the first period of time has elapsed. Depending on polymer strength, the mesh may not be required. The polymer solution is then electrospun onto the mandrel for a second period of time, wherein the second period of time is an amount of time required to embed the polyester braid into the electrospun polymer to form the fabricated textile. The residual organic solvent is removed from the fabricated textile and the fabricated textile is removed from the mandrel. The tissue cuff is formed from the fabricated textile. The tissue cuff comprises a first fastening portion defining one or more openings, one or more bands extending from the first fastening portion at a first end to an opposing second end, and a second fastening portion positioned at the opposing second end of the one or more bands. The tissue cuff is then formed from the electrospun polyester. This process allows for modification of the final parameters and consistent reproduction of the device once a final structure is accepted. The forming of the tissue cuff may be performed by a variety of processes to yield a reproducible, accurate result including, but not limited to, laser cutting and die punch cutting.

An embodiment of a tissue compression cuff comprises a first fastening portion defining one or more openings and one or more bands comprising a first end that extending from the first fastening portion at a first end, and an opposing second end. The one or more bands define a first diameter at the first end and a second diameter at the second end, wherein the first diameter is greater than the second diameter. The tissue compression cuff further comprises a second fastening portion positioned at the opposing second end of the one or more bands. The second end of the one or more bands is configured to be inserted through the one or more openings of the first fastening portion. The tissue compression cuff is comprised of an electrospun textile comprising biocompatible nanofibers.

An embodiment of a fistula cuff comprises a first fastening portion defining one or more openings and at least one band comprising a first end extending from the first fastening portion at a first end, and an opposing second end. The at least one band defines a first diameter at the first end and a second diameter at the second end such that the first diameter being larger than the second diameter. A second fastening portion is positioned at the opposing second end of the at least one band. The second end of the at least one band is configured to be inserted through the one or more openings of the first fastening portion. The tissue compression cuff is sized and configured to reduce blood flow through an arteriovenous fistula and made from an electrospun textile made up of biocompatible nanofibers.

### BRIEF DESCRIPTION OF THE DRAWINGS

A more particular description of the invention briefly summarized above may be had by reference to the embodiments, some of which are illustrated in the accompanying drawings. It is to be noted, however, that the appended drawings illustrate only typical embodiments of this invention and are therefore not to be considered limiting of its scope, for the invention may admit to other equally effective embodiments. Thus, for further understanding of the nature and objects of the invention, references can be made to the following detailed description, read in connection with the drawings in which:

FIG. 1 illustrates a front perspective view of an embodiment of an electrospinning apparatus;

FIG. 2 is a plan view of a tissue cuff made in accordance with a first embodiment;

FIG. 3 is a plan view of a tissue cuff made in accordance with a second embodiment;

FIG. 4 is a plan view of the tissue cuffs of FIGS. 2 and 3;

FIGS. 5(A) and 5(B) detail assembly views of a tissue cuff made in accordance with aspects of the invention as placed and secured to a simulated fistula, as represented by a balloon;

FIGS. 6 (A), (B) and (C) are scanning electron microscopy (SEM) images detailing surface morphology of tissue cuffs made in accordance with aspects of the invention;

FIGS. 7(A) and 7(B) present a comparative histological assessment of cell compatibility between a known control sample (FIG. 7(A)) and an electrospun material (FIG. 7(B)); and

FIGS. 8(A) and 8(B) are comparative immunohistological images taken in vivo between woven and electrospun polyester materials.

### DETAILED DESCRIPTION

The following description relates to various embodiments of an improved tissue cuff made in accordance with electrospinning technology. It will be readily apparent that these embodiments are merely examples and that numerous variations and modifications are possible that embody the inventive aspects discussed herein. Although the foregoing description is limited to applications of the tissue cuff for AVF constriction, the inventive concepts described herein are also applicable to other intravenous apparatus. Several terms are used throughout this description to describe the salient features of the invention in conjunction with the accompanying figures. These terms, which may include “first”, “second”, “inner”, “outer”, and the like are not intended to overly limit the scope of the invention, unless so specifically indicated. The terms “about” or “approximately” as used herein may refer to a range of 80%-125% of the claimed or disclosed value. With regard to the drawings, their purpose is to depict salient features of the pump assembly and are not specifically provided to scale.

Disclosed herein is a nanofibrous material construct, which is manufactured either in tubular or flat sheet form using a unique electrospinning perfusion methodology. One particular embodiment provides a nanofibrous composite (or biocomposite) material formed as a discrete textile fabric from a prepared liquid admixture of: (i) a biodurable synthetic polymer; and (ii) a liquid organic carrier. In some embodiments, a biologically active agent is included as part of the admixture. The liquid admixture can be prepared using diverse compositions and is employed in a novel electrospinning perfusion process to form a textile comprised of nanofibrous material, which in turn, can serve as the antecedent precursor and tangible workpiece for subsequently making the desired medical article or device suitable for use in-vivo. Prior art for medical devices generally includes an underlying non-polymeric support (e.g. scaffold, or stent) coated with a biodegradable polymer and/or in some instances, soaked in a biologically-active agent to embed the agent in the polymer. In contrast, the tissue cuffs of the present invention are discrete articles that omit the underlying scaffold and consist essentially of a non-biodegradable polymer that may further comprise a biologically-active agent embedded therein. The materials of the present invention have mechanical properties which are sufficient to permit the manufacturer to omit the scaffolds that were previously required by the prior art.

In embodiments of the tissue cuff that are formed using an admixture comprising one or more biologically active agents, the one or more biologically-active agents will have become non-permanently immobilized and releasably bound to the tangible nanofibrous material of the fabricated textile of the tissue cuff. These non-permanently immobilized biologically-active agents are well established chemical compounds that retain their recognized biological activity both before and after becoming impermanently (i.e., temporarily or reversibly) bound to the textile fabric of the tissue cuff; and will become subsequently released in-situ and directly delivered into the ambient environment as discrete mobile entities when the textile fabric of the tissue cuff takes up any fluid—i.e., any aqueous or organic based liquid. Accordingly, via the transitory immobilization of one or more biologically active molecules to the nanofibrous biocomposite material, the agent-releasing textile is very suitable for inclusion and use in-vivo as a clinical/therapeutic construct. Examples of biologically active agents include, but is not limited to, antimicrobials, anticoagulants, analgesics, antiplatelets, steroidals, anti-inflammatories, anti-proliferatives, and cancer drugs.

In embodiments of the tissue cuff that are formed using an admixture comprising one or more compounds or active agents, the one or more compounds or active agents will have become permanently immobilized into the tangible nanofibrous material of the fabricated textile of the tissue cuff. These permanently immobilized agents are well established chemical compounds that retain their ability to be seen using various imaging procedures before and after becoming permanently bound to the textile fabric of the tissue cuff. Accordingly, via the transitory immobilization of one or more active molecules to the nanofibrous biocomposite material, the agent-releasing textile is very suitable for inclusion and use in-vivo as a clinical/therapeutic construct. Examples of these compounds or active agents include, but is not limited to, radiopaque and fluoroscopy imaging agents such as gadodiamide, ditrizoic acid and barium sulfate. The present electrospinning perfusion method of making nanofibrous textiles that may comprise releasable biologically active agents has several major advantages and desirable benefits to the commercial manufacturer, as well as to the physician and surgeon. Among these benefits are the following:

First, the manufacturing methodology comprising the present invention does not utilize any immersion techniques and does not require submerging the fabricated textile in any immersion baths, soaking tanks, or dipping pools for any purpose. Rather, the methodology preferably utilizes the unique technique of electrospinning perfusion as a manufacturing method in order to blend a synthetic substance and a biologically active agent of choice together as a fabricated textile.

Second, the electrospinning perfusion method of manufacture yields a fabricated textile having particular characteristics. The fabricated textile can be folded, or twisted, and otherwise manipulated to meet specific requirements of thickness, gauge, or deniers; and can also be cut, split, tailored, and conformed to meet particular shapes, configurations and patterns.

Third, the fabricated textile is a nanofibrous material composite comprised of multiple fibers, has a determinable individual fiber thickness in or near the nanometer size range (typically not more than 3 microns), and can present a discernible fiber organization and distribution pattern. These fabricated textiles provide and demonstrate excellent suture retention, burst strength, break strength, tear strength and/or biodurability.

Fourth, the manufacturing method comprising the present invention may employ limited heat and compression force to alter the exterior surface of the fabricated textile originally formed via the electrospinning perfusion technique. This exterior surface treatment portion of the manufacturing process is optional, but when employed, will produce a highly desirable fixed exterior surface over the entire linear length of the fabricated textile article.

Fifth, the biologically active agent will retain its characteristic biological activity both before and after being temporarily bound to the nanofibrous material. The attributes and properties associated with the biologically active agent of choice will co-exist with and be an integrated feature of the resulting textile article at the time the article is utilized.

The method of the present invention is directed in part to the making of an agent-releasing textile, an antecedent article of manufacture, which is then employed as a tangible workpiece to generate a subsequently prepared medical article or device suitable for use in-vivo. An “agent-releasing textile” as defined herein is a fabricated textile comprising nanofibrous matter which has at least one biologically active agent immobilized onto and/or within the material substance of the textile; and which, upon wetting, is then able to release the biologically active agent in-situ and deliver it in a functionally operative form into the adjacent local area or immediately surrounding environment. Such a prepared nanofibrous textile must provide and release at least one active chemical composition, compound, or molecule which is active, functional and operative either to influence and/or to initiate or cause a recognizable pharmacological effect or determinable physiological change in the living cells, tissues and organs of the host patient. A “fabricated textile” as defined herein is an article of manufacture which is comprised, in whole or in part, of fibers arranged as a fabric. The fibers comprising the fabricated textile may be chosen from a diverse range of organic synthetics, prepared polymer compounds, or naturally-occurring matter. In general, the fabricated textile is often prepared as a cloth or fabric; and may comprise a single fiber film, or a single layer of fibrous matter; multiple layers of fibrous matter; or exist as multiple and different deniers of fibers which are present in a range of varying thickness, dimensions, and configurations.

It will be appreciated that, after the agent-releasing nanofibrous textile has been manufactured and is present as a discrete entity, it can optionally serve as a tangible workpiece in combination with other items and additional components and hardware to yield the desired end product, a clinically or therapeutically useful “medical article or device”. Thus, regardless of its true chemical composition/formulation or the particular mode of construction, the initially formed “agent-releasing textile” and the subsequently generated “medical article or device” are directly and intimately related.

By definitional requirement, the agent-releasing nanofibrous textile (optionally also the antecedent forerunner of each subsequently generated medical article or device) is a non-woven material comprised of discrete fibers. The nanofibrous composite material forming the textile fabric has been electrospun from a liquid admixture and blending in a liquid organic carrier of at least one synthetic substance (and optionally at least one biologically active agent). This admixture can be prepared in a wide range of varying ratios using a liquid organic carrier, followed by application of an electric current to create the biocomposite material.

To illustrate the range and variety of compositions deemed suitable for use as a blended mixture, a listing of suitable synthetic substances is presented in the following Table 1. It will be noted that the listings in Table 1 present a number of exemplary synthetic substances long deemed suitable for use alone or in blends to produce synthetic fibers. To complete the description, Table 2 lists some of the typical and more commonly available organic liquids that can be usefully employed alone and/or in blends as the liquid carriers.

TABLE 1
Illustrative Synthetic Substances

Polymeric Fibers
polyester
polyethylene terephthalate; poly(butylene) terephthalate;
poly(trimethylene) terephthalate
Polyurethane;
polyglycolic acid;
polyamides, including nylons and aramids;
Polytetrafluoroethylene; and
mixtures of these substances
Other synthetic fiber compositions (using TFPIA generic fiber names)
Acetate;
Triacetate;
Acrylic;
Modacrylic;
Olefin (Polypropylene, polyethylene, and other polyolefins);
Saran

TABLE 2
Representative Organic Liquid Carriers

Hexafluoroisopropanol;
Dimethylformamide;
Dimethylsulfoxide;
Acetonitrile;
Acetone;
Methanol;
Hexamethylphosphoric triamide;
N,N-diethylacetamine;
N-methylpyrrolidinone;
Ethanol;
4-methylmorpholine-N-oxide monohydrate

At least some of the fibers comprising the textile fabric will demonstrate a range of properties and characteristics, for example:

1. The fibers constituting the agent-releasing textile (and the subsequently generated medical article or device) will have a demonstrable capacity to take up water and/or aqueous liquids and/or organic liquids and/or organic based liquids (with or without direct wetting of the fibrous material). The mode or mechanism of action by which organic and aqueous fluids are taken up by the fibers of the textile (and/or become wetted by the fluid) is technically insignificant and functionally meaningless. Thus, among the different possibilities of fluid (aqueous and/or organic) uptake are the individual alternatives of: absorption; adsorption; cohesion; adhesion; covalent bonding; non-covalent bonding; hydrogen bonding; miscible envelopment; molecule entrapment; solution-uptake between fibers; fiber wetting; as well as others well documented in the scientific literature. Any and/or all of these may contribute to organic and/or aqueous fluid uptake in whole or in part. Which mechanism of action among these is actively in effect in any instance or embodiment is irrelevant.

2. By choosing a particular chemical formulation and/or desired stereoscopic (or three-dimensional) structure for the synthetic substance of the fabrication, the resulting biologically active textile can be prepared as a fabric having a markedly long functional duration and lifespan for in-vivo use. Accordingly, by choosing one or more durable and highly resilient chemical compositions as the fibers of choice, textiles effective for many years' duration and utility may be routinely made. All of these choices and alternatives are conventionally known and commonly used today by practitioners in this field.

It is also well recognized that some synthetic chemical compositions are available in a range of diverse formulations. One example of a highly resistant chemical composition having many alternative formulations is poly(ethylene terephthalate), of which one particular formulation is sold under the trademark DACRON®. As is commonly known in this field, a range of differently formulated polyethylene terephthalates (or “PETs”) are known to exist and are commercially available, each of these alternatives having a different intrinsic viscosity [or “IV”, as measured in o-chlorophenol or “OCP”, at 25° C.]. Typically, these differently formulated polyethylene terephthalate compounds can vary from less than 0.6 dl/g [IV] to greater than 1 dl/g [IV]; yet each of these alternative polyethylene terephthalate formulations can be dissolved in ice-cold 100% hexafluoroisopropanol (“HFIP”). Thus, the electrospinning of appropriately prepared HFIP solutions containing any of such alternatively formulated polyethylene terephthalates will result in the fabrication of nanofibrous textile fabrics which are capable of independent or combined release of many diverse drugs, proteins and genetic materials.

3. The fibers comprising the agent-releasing textile (and the subsequently generated medical article or device) can be prepared in a variety of organizations as a tangible structure. Thus, as conventionally recognized within the textile industry, the textile fabric may vary in size or thickness; and may optionally receive one or more interior and/or exterior surface treatments to enhance particular attributes such as increased in-vivo biocompatibility or a greater expected time for functional operation and use in-vivo. All of these organizational variances are deemed to be routine matters which will be optionally chosen and desirably used to meet particular medical needs or individual patient requirements.

4. The fibers comprising the agent-releasing textile (and the subsequently generated medical articles or devices) can be prepared to meet the particulars of the intended in-vivo medical use circumstances or the contingencies of the envisioned clinical/therapeutic application. Thus, the textile fabric can alternatively be prepared either as a relatively thin-walled biocomposite, or alternatively as a thick-walled material; be produced as an elongated object having a diverse range of different outer diameter and inner diameter sizes; and be fashioned as a relatively inflexible or unyielding item, or as a very flexible and easily contorted length of matter.

### The Choosing of an Appropriate Biologically Active Agent

A number of different biologically active agents can be beneficially and advantageously utilized in tandem with the nanofibrous textile fabric. However, there are several minimal requirements and qualifications which the biologically active molecule—whatever its particular composition and formulation as a chemical compound, composition or molecule—must demonstrably provide in order to be suitable for use in the present invention. These are:

(i) The chosen agent must be capable of demonstrating its characteristic biological activity before becoming temporarily bound to and immobilized by the material substance of the fabricated textile. This characteristic biological activity must be well recognized and will constitute its ability/capacity to function as an active mediator in-situ.

(ii) The particular agent immobilized upon or within the material substance of the textile fabric must be capable of demonstrating its characteristic biological activity (its mediating capacity) after becoming immobilized and bound; and

(iii) The immobilized agent bound into the material substance of the textile fabric will be released in-situ from the non-biodegradable polymer and be delivered into the surrounding local environment as a freely mobile molecule that retains its characteristic biological activity (its mediating capacity) over an extended period of time after the agent-releasing textile has been utilized in-vivo and allowed to take up water.

In addition, since the primary medical application for the fabricated textile is expected to differ and vary extensively from one embodiment to another, it is intended that the characteristic biological properties of the chosen agent serve to aid, promote, and/or protect the naturally occurring pathways and processes of the body which occur in-vivo.

Accordingly, it is deemed likely that the primary function and capabilities of the chosen biologically active molecule will differ and vary in many instances; and thus there are multiple purposes and a range of individual goals for the releasable substance, among which are the following: (1) to serve as an antimicrobial agent—i.e., as an anti-bacterial or anti-fungal composition having a broad or narrow spectrum of activity; (2) to function as an anti-neoplastic compound effective against specific kinds of tumors; (3) to operate as a selective physiological aid—i.e., as a mediator which serves to avoid vascular complications such as blood coagulation or acts to prevent the formation of blood clots; (4) to act as a pharmacological composition—i.e., as a drug or pharmaceutical which deactivates specific types of cells and/or functions to suppress or inhibit a variety of different humoral and cellular responses associated with or related to inflammation and the inflammatory response in-vivo; and (5) to function as a biological indicator or contrasting agent. Examples of each are presented hereinafter.

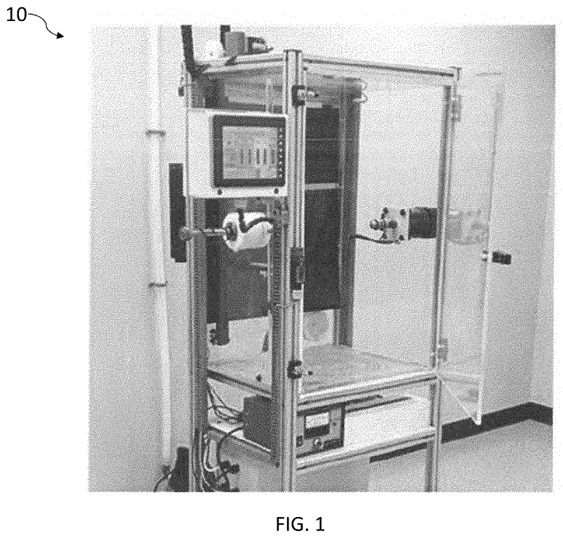

A preferred method for making the agent-releasing textile of the present invention is via the unique technique of electrospinning perfusion, either on a large scale electrospinning apparatus (as shown in FIG. 1) or in small batches. For this purpose, an electrospinning perfusion assembly or apparatus is erected which comprises, at a minimum, a collecting surface such as a rotating mandrel with a target surface which can be set at a pre-selected rotation speed; a needle fronted perfusion instrument with a spinerette, such as a syringe, which can be set to deliver a liquid mixture at a pre-specified flow rate; an electrical coupling for controlling and coordinating the electrical voltage applied across the perfusion needle and which is grounded to the rotating mandrel; and a controllable supply of electrical power.

An admixture is prepared comprising blending an organic solvent or organic liquid carrier and 5-30% w:v of at least one non-biodegradable polymer (and optionally at least one biologically active agent). In one embodiment, the organic liquid carrier is cooled to an ice-cold (e.g. between about −10° C. and about 5° C.) temperature. For reasons that are not clear, this cooling step facilities the proper formation of the admixture and speeds the dissolution of the non-biodegradable material.

For example, a 10 ml syringe with a stainless steel 18-gauge blunt spinneret (0.5 mm internal diameter) is then filled with the polymer admixture and placed onto a syringe pump, such as a Harvard Apparatus syringe pump, for subsequent perfusion. Perfusion is the action and the act of causing a liquid or other fluid to pass across the external surfaces of, or to permeate through, the substance of a tangible entity or a configured physical construct. Perfusion of a liquid or fluid thus includes the alternative actions of: a sprinkling, pouring, or diffusing through or overlaying action; a covering, spreading, penetrating or saturating action (termed “suffusion”); a slow injection or other gradual introduction of fluid into a configured space or sized internal volume (termed “infusion”); and a passage across a surface or through a discrete surface or tangible thickness of matter, regardless of the mechanism or manner of transfer employed for such fluid passage.

Once the admixture has been properly loaded, the electrical coupling and syringe pump are activated and the admixture is electrospun onto the target surface. In one embodiment, the step of electrospinning is carried out at a temperature which does not harm the biological activity of the biologically-active agent in the admixture. The reaction temperature is, in one embodiment, ambient room temperature (20-25° C.), but when necessary or desired can be chosen to be within a temperature reaction range of about 0-50° C.

Utilization of this assembly permits uniform coating of the liquid admixture onto the surface of the mandrel; and the applied electrical voltage can be varied as needed to control the formation of the nanofibers upon the mandrel's surface.

It will be recognized in particular that electrospinning over a broad range of conditions is possible for polyesters. Thus, a range of differently formulated polyethylene terephthalates (or “PETs”) of intrinsic viscosity [or “IV” as measured in OCP at 25° C.] that range from less than 0.6 dl/g [IV] to greater than 1 dl/g [IV] can be dissolved in ice-cold 100% hexafluoroisopropanol (“HFIP”). Electrospinning appropriately prepared HFIP solutions of such polyethylene terephthalates results in the fabrication of nanofibrous textile fabrics capable of independent or combined release of diverse drugs, proteins and genetic materials.

### Perfusion

Perfusion of the polymer solution begins upon application of the electric current to the tip of the syringe needle (typically 15-30 kV), which then moves at a preset constant speed and fixed distance from the mandrel surface for a pre-determined time period to create materials of varying thicknesses. This process of manufacture is therefore termed “electrospinning perfusion”; and yields a fully fabricated, elongated nanofibrous textile whose final flat sheet dimensions correspond to the overall outer diameter of the mandrel (in this instance, 30 mm).

Multiple nozzles (or syringe needles) can be used concurrently to reduce the time required to fabricate structures of the appropriate rigidity. The use of multiple injection streams to increase production rates is a familiar concept to those skilled in the art; and, accordingly, the use of multiple nozzles lies within the scope of the present invention.

### Production of Electrospun Fabricated Textile

Similar in its essentials to the technique described above, polymer chips were dissolved in an organic solvent at a temperature of from about −10° C. to about 5° C. In an embodiment the solvent used is 100% hexafluoroisopropanol (“HFIP”) and the polymer is polyester, however any combination of the polymers and solvents disclosed may be used. The organic solvent and polymer is mixed on an inversion mixer for 48 hours in order completely solubilize the polymer. If a biologically active agent is used, it is added to the organic solvent along with the polymer. The self-contained, semi-automated electrospinning apparatus containing a Glassman power supply, a Harvard Apparatus syringe pump, an elevated holding rack, a modified polyethylene chamber, a spray head with power attachment and a reciprocating system was again used. An example of an electrospinning apparatus is shown in FIG. 1. The apparatus includes a mandrel for accepting the electrospun polymer.

A 10 ml chemical-resistant syringe was filled with the polymer liquid. A stainless steel 18-gauge blunt spinneret (0.5 mm internal diameter) was then cut in half, with the syringe fitting end connected to the polymer-filled syringe. Teflon® tubing was connected to the syringe filled with the polymer solution followed by connection to the other half of the blunt spinneret within the spray head. The line was then purged of air, with the syringe then placed onto the syringe pump. The high potential source was connected to the spray head tip, with the plate set at a jet gap distance of 15 cm (range of 5 cm-50 cm) from the tip of the needle. The perfusion rate can be set at 1-20 ml/hour at 25° C.

Perfusion of the polymer liquid was started upon application of the current to the tip of the needle (15-30 kV) with electrospinning proceeding for a first period of time, for example 30-120 minutes, with rotation of the mandrel at a rate of from 10-1000 r.p.m. The mandrel may mandrel may be comprised of any suitable material such as stainless steel and may be coated by one or more substances, such as Teflon®, to aid in the removal of the electrospun material or electrospun polymer from the mandrel. After the first period of time has elapsed, a polymer yarn mesh, for example a polyester yarn mesh, is braided onto the electrospun polymer using a 16 carrier braider. In an embodiment, 2-16 polymer yarn meshes are braided onto the electrospun polymer. The mandrel is then reinserted into the electrospinning apparatus and the electrospinning is resumed for a second period time, for example 52-252 minutes, until the polymer yarn mesh braid is embedded into the electrospun polymer and the desired thickness is achieved. The electrospun material is then removed from the mandrel resulting in a flat, generally planar sheet of nanofibrous textile material being formed. The resulting sheet of fabricated textile comprises nanofibers having a diameter no greater than about 3 microns and preferably from approximately 0.8 microns to approximately 3 microns. In other embodiments, the diameter of the nanofibers is between 200 nm-2000 nm. The sheet of fabricated material is removed from the mandrel, positioned flat and then laser cut into the desired shape. As described below, the fabricated textile material is formed into a tissue cuff using laser cutting, die punch cutting, or any other suitable method to reproducibly and accurately form a tissue cuff shape from the fabricated textile.

In an embodiment, an admixture was prepared comprising blending an organic solvent or organic liquid carrier. Poly(ethylene terephthalate) (15% w:v) and poly(butylene terephthalate) (2% w:v) were prepared in HFIP and mixed until polymer chips were dissolved. The polymer was placed into a syringe and placed into the electrospinning unit. The high potential source was connected to the spray head tip, with the 30 mm mandrel set at a jet gap distance of 15 cm from the tip of the needle. The perfusion rate was set at 6 ml/hour at 25° C.

Perfusion of the polymer liquid was started upon application of the current to the tip of the needle (20 kV) with electrospinning proceeding for about 60 minutes with rotation of the mandrel at a rate of from 280 r.p.m. After the first period of time had elapsed, a polymer yarn mesh, for example a polyester yarn mesh, was braided onto the electrospun polymer using a 16 carrier braider, with 8 polyester yarns (100-300 denier yarns) braided onto the electrospun polymer. The mandrel was then reinserted into the electrospinning apparatus and the electrospinning is resumed for about 110 minutes, until the polymer yarn mesh braid is embedded into the electrospun polymer and the desired thickness is attained. In an embodiment, the resulting tissue cuff may comprise a thickness of approximately 200 μm-1 mm, however the resulting thickness may vary based on alteration of one or more of the electrospinning parameters discussed in order to produce a tissue cuff with the desired properties.

Different embodiments may comprise an electrospun polymer material of a different thickness and/or strength, using braids of different composition (e.g., increased number of braids, increased yarn thickness, increased tensile strength) and device configuration (e.g., smaller profile, increased length for ease of application).

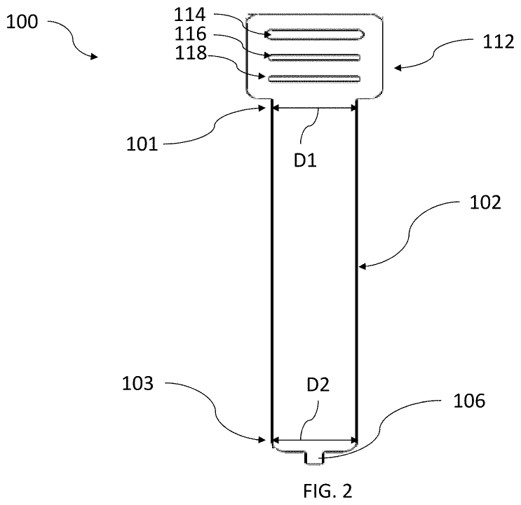

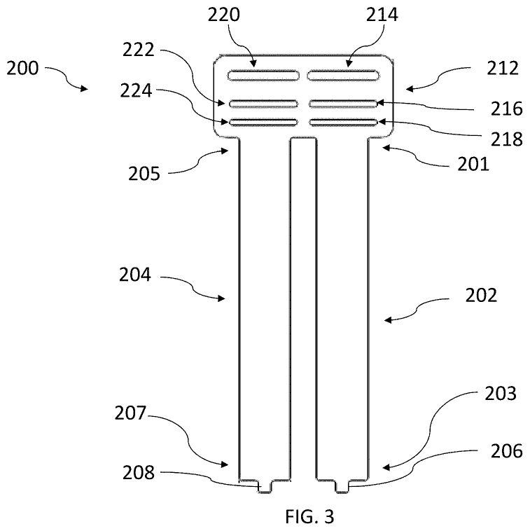

For purposes of this discussion, two (2) tissue cuffs made in accordance with embodiments of the invention are shown in FIGS. 2-4. FIG. 2 depicts a single-arm or single-band tissue cuff 100, while FIG. 3 depicts a dual-arm version 200. While the tissue cuffs disclosed may be used to surround, compress or otherwise decrease the volume of a biological conduit/pouch (such as a stomach in a case of a gastric band), the below discussion pertains to an embodiment of the tissue cuff shown in FIGS. 2-4 that may be used as a fistula cuff having excellent handling, good gross strength and flexibility. The fistula cuffs 100, 200 are configured to reduce blood flow at a single point on the AVF near the anastomotic region, however in other embodiments the tissue cuff may be used to constrict an air passage, a gland duct, or any other biological conduit.

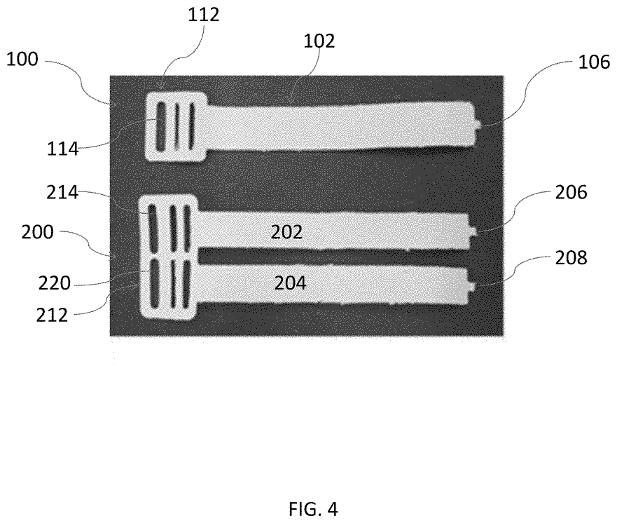

As shown in FIGS. 2-4, the fistula cuffs 100, 200 are shown in an unengaged state. In the unengaged state, the fistula cuffs 100, 200 are generally planar. Referring to FIGS. 2 and 4, the fistula cuff 100 includes a band 102 that extends from a first fastening portion 112 at a first end 101 and a second fastening portion 106 at an opposing end 103. The first fastening portion 112 may include one or more slits or openings that are dimensioned to receive and secure the opposing end 103 of the band 102. As shown, the fistula cuff 100 defines three (3) slits or openings 114, 116, 118 in which opening 114 is larger than an adjacent opening 116, which is in turn larger than another adjacent opening 118. In another embodiment, two or more of the slits or openings 114, 116, 118 are the same size. In an embodiment, the first end 101 of the band 102 may comprise a first diameter D1 and the second end 103 of the band 102 may comprise a second diameter D2, such that D1>D2. In a further embodiment, the band 102 may be generally tapered from the first diameter D1 to the second diameter D2 along the band 102.

Referring to the embodiment of the fistula cuff 200 shown in FIGS. 3 and 4, two adjacent bands 202, 204 are provided, which are each coupled to a first fastening portion 212 at one end 201, 205 and having a second fastening portion 206, 208 on an opposing end 203, 207. The first fastening portion 212 includes two or more slits or openings 214, 216, 218, 220, 222, 224, that are dimensioned and shaped to receive and secure the opposing end of the respective band 202, 204. As shown in FIGS. 3 and 4, the fistula cuff 200 has six (6) slits or openings that are arranged in two (2) groups of three (3) adjacent openings. According to this embodiment, openings 214 and 220 are larger than openings 216 and 222, which are in turn larger than openings 218 and 224. In another embodiment, three or more of the openings 214, 216, 218, 220, 222, 224 can be the same size. It will be readily apparent that the number of bands (single, dual) of the cuff can be varied, as well as the number of openings or slits provided. In an embodiment, one or more of the bands may be a different length.

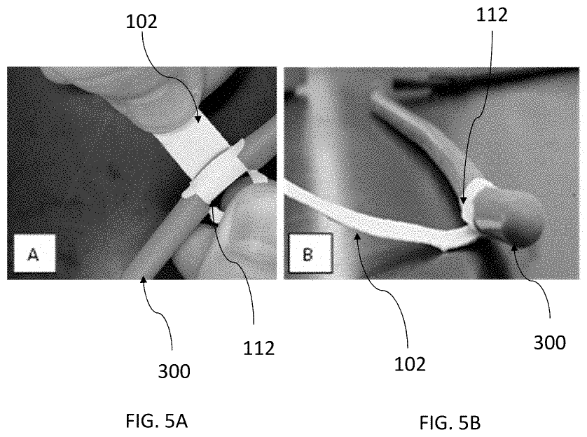

Referring to the embodiment in FIG. 2 and FIGS. 5(A)-(B), and in the unengaged state the fistula cuff 100 may be positioned so that at least a portion of the fistula cuff 100 is located under the AVF. A balloon 300 is used in place of an in vivo AVF for the purposes of illustration. The end of the band 102 having the second fastening portion 106 is brought around to encircle the AVF and is pulled through the opening 114, which causes the band 102 to exert a compressive force on the AVF in the radial direction, which constricts the AVF. The band 102 may be pulled through the opening 114 until the desired compressive force exerted by the band 102 on the AVF is achieved. The end 106 of the band 100 having the second fastening portion 106 may then be looped back and pulled through one or more of the remaining adjacent openings 116, 118 in order to secure the fistula cuff 100 in position while maintaining the desired compressive force. The constriction or compressive force remains due to the fabricated polymer textile holding itself. The second fastening portion 106 may be dimensioned to aid threading of the free end of the band 102 through the openings 114, 116, 118. In an embodiment, the second fastening portion 106 may comprise a taper or a tab that is not as wide as the band 102 and therefore, may be more easily threaded through the openings 114, 116, 118. The fistula cuff 100 may additionally be sutured to itself or surrounding tissue to further prevent movement of the fistula cuff 100.

Referring to the embodiment shown in FIG. 3, the fistula cuff 200 is placed on the AVF in a similar manner as the embodiment shown in FIGS. 2 and 5A-B, however the use of multiple bands 202, 204 allows for each band 202, 204 to exert a different amount of compressive force on the AVF. For example, band 202 may be pulled through openings 214, 216, and 218 to exert a compressive force F on the AVF and the band 204 may be pulled through openings 220, 222, and 224 to exert compressive force F′ on the AV fistula, where F>F′. In this manner, the fistula cuff 200 can exert a graduated compressive force on the AVF.

Although the foregoing description is limited to applications of the fistula cuff 100, 200 for AVF constriction, other embodiments of the fistula cuff 100, 200 may be used to constrict a conduit other than an AVF, such as a native artery (pulmonary artery) or vein. In an embodiment, the fistula cuff 100, 200 may be used to constrict an air passage, a gland duct, or any other biological conduit. In another embodiment, the fistula cuff 100, 200 may be used to surround and/or secure biological tissue for drug delivery and/or to promote healing. In a further embodiment, the fistula cuff 100, 200 may be used to secure another medical implant or drug delivery device in the body.

### Surface and Physical Strength Characterization

### Surface Morphology Via Scanning Electron Microscopy (SEM):

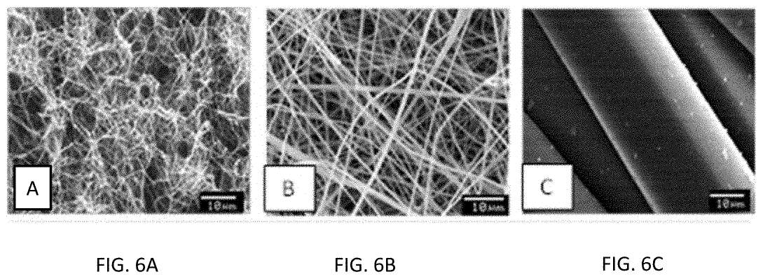

Referring to FIGS. 6(A)-(C), SEM images for the polyester material comprising the electrospun fistula plug revealed a fibrous structure similar to extracellular matrix (ECM). By varying the electrospinning parameters, meshes with fiber diameters ranging from 200 nm to 3 μm can be produced. In other preferred embodiments, the electrospun fistula cuff 100, 200 has fibers ranging between 0.8-3 μm. Fiber diameters below 5 μm have been shown to reduce fibrotic response. As shown generally in FIGS. 6(A)-(B), the orientation of these fibers was asymmetric throughout the material, similar to ECM.

### Physical Testing:

Tensile strength (break strength) for the single band electrospun fistula cuff 100 were assessed using a Q-Test apparatus. Each end of the fistula cuff 100 was placed into clamps that were spaced 1 cm apart. Stretching of the fistula cuff 100 (pull rate 200 mm/min, load cell=25 pounds) was then initiated and terminated upon segment breakage. The results showed that 4.8 pounds force (2.17 kgF) was required to break the fistula cuff 100. This indicates that the fistula cuff 100 possesses significant strength to withstand pull forces that will be required to implant the fistula cuff 100.

### In Vitro Biocompatibility Studies

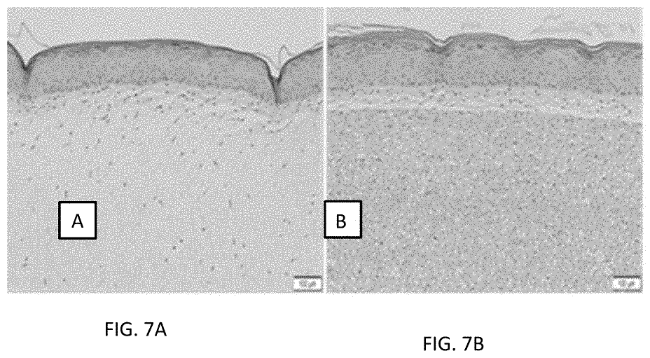

The electrospun polyester material comprising the fistula cuff 100, 200 has been evaluated for cell compatibility in various tissue culture studies. In one example, the electrospun polyester material was evaluated as a scaffold to grow human tissue, in this case skin (fibroblasts and keratinocytes). Referring to FIGS. 7(A)-(B), a scaffold containing rat collagen was used as a positive control (FIG. 7(A)) and the electrospun polyester material without any proteins was the test material (FIG. 7(B)). Fibroblast growth throughout the electrospun polyester material was comparable to the protein-coated scaffold. Keratinocyte growth onto the fibroblasts was also comparable. This demonstrates that the electrospun polyester material scaffold has comparable cell growth to a protein-based scaffold.

### In Vivo Implantation Data

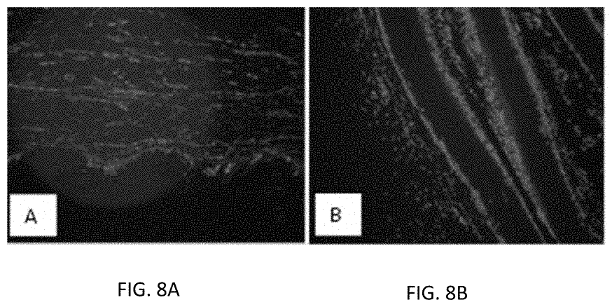

The electrospun polyester material has been implanted in various preclinical models, from small (mice/rats) though large (canines) animals. Regardless of the preclinical model, these materials have shown excellent tissue incorporation and healing after implantation. One model that directly compared healing was the rat dorsal subcutaneous model in which woven and electrospun polyester materials were implanted for 4 months (FIGS. 8(A)-(B)). The electrospun polyester material was further tested for capillary presence via Isolectin staining. Woven polyester (FIG. 8(A)), while having some capillaries present around the material, had minimal capillaries within the material. In stark contrast, electrospun polyester materials (FIG. 8(B)) had grossly more capillaries around and within the material. This type of cellular ingrowth may allow the electrospun fistula cuff 100, 200 to fully integrate into the surrounding tissue thereby preventing any release failures.

Additional embodiments include any one of the embodiments described above and described in any and all exhibits and other materials submitted herewith, where one or more of its components, functionalities or structures is interchanged with, replaced by or augmented by one or more of the components, functionalities or structures of a different embodiment described above.

It should be understood that various changes and modifications to the embodiments described herein will be apparent to those skilled in the art. Such changes and modifications can be made without departing from the spirit and scope of the present disclosure and without diminishing its intended advantages.

Although several embodiments of the disclosure have been disclosed in the foregoing specification, it is understood by those skilled in the art that many modifications and other embodiments of the disclosure will come to mind to which the disclosure pertains, having the benefit of the teaching presented in the foregoing description and associated drawings. It is thus understood that the disclosure is not limited to the specific embodiments disclosed herein. Moreover, although specific terms are employed herein, they are used only in a generic and descriptive sense, and not for the purposes of limiting the present disclosure.

1. A method of forming a tissue cuff from a fabricated textile comprised of electrospun nanofibers, the method comprising:
dissolving a non-biodegradable polymer in an organic solvent to produce a polymer solution;
loading the polymer solution into an electrospinning instrument configured to be set at a specified flow rate of 1-20 ml/hr.;
placing the mandrel from the needle tip at a distance ranging from 5 cm-50 cm;
applying an electric current of 15-30 kV to a needle of the electrospinning instrument;
electrospinning the polymer solution onto a mandrel for a first period of time to form an electrospun polymer material, the electrospinning occurring at room temperature and the mandrel rotating at 10-1000 r.p.m.;
braiding a polyester yarn mesh onto the electrospun polymer material on the mandrel after the first period of time has elapsed;
electrospinning the polymer solution onto the mandrel for a second period of time, wherein the second period of time comprises an amount of time required to embed the polyester braid into the electrospun polymer to form the fabricated textile material;
removing residual organic solvent from the fabricated textile;
removing the fabricated textile from the mandrel; and
forming the tissue cuff from the fabricated textile, wherein the tissue cuff comprises:
a first fastening portion defining one or more openings,
one or more bands extending from the first fastening portion at a first end to an opposing second end, and
a second fastening portion positioned at the opposing second end of the one or more bands.

2. The method of claim 1, wherein the forming of the tissue cuff from the fabricated textile comprises at least one of laser cutting and die punching of the fabricated textile.

3. The method of claim 1, wherein the one or more openings of the first fastening portion are configured to accept the second end of the one or more bands.

4. The method of claim 1, wherein the polymer solution comprises at least one biologically active agent.

5. The method of claim 4, wherein the at least one biologically active agent comprises at least one of an antimicrobial agent, an anti-proliferative agent, an anti-inflammatory agent, a steroid, and a cancer drug.

6. The method of claim 4, wherein the at least one biologically active agent comprises at least one of a radiopaque and a contrast agent.

7. The method of claim 1, wherein the polymer solution comprises 5-30% weight per volume of the polymer.

8. The method of claim 1, wherein the nanofibers are biocompatible and comprise a diameter of from about 0.5 μm to about 3 μm.

9. The method of claim 7, wherein the fabricated textile comprises a mesh.

10. The method of claim 1, wherein the electrospinning comprises a first specified flow rate during the first period of time and a second specified flow rate during the second period of time, wherein the first specified flow rate is different than the second specified flow rate.

11. The method of claim 1, in which the tissue cuff is sized and configured to reduce blood flow through an arteriovenous fistula of a patient during hemodialysis.

12. The method of claim 1, wherein the non-biodegradable polymer comprises at least one of polyester and polyurethane.

13. A tissue compression cuff comprising:
a first fastening portion defining one or more openings;
one or more bands comprising a first end that extending from the first fastening portion at a first end, and an opposing second end, the one or more bands defining a first diameter at the first end and a second diameter at the second end, wherein the first diameter is greater than the second diameter; and
a second fastening portion positioned at the opposing second end of the one or more bands, wherein the second end of the one or more bands is configured to be inserted through the one or more openings of the first fastening portion,
wherein the tissue compression cuff is comprised of an electrospun textile comprising biocompatible nanofibers.

14. The tissue compression cuff of claim 13, wherein the biocompatible nanofibers comprise a diameter of from 200 nm to about 2000 nm.

15. The tissue compression cuff of claim 13, wherein the electrospun textile comprises a biologically active agent.

16. The tissue compression cuff of claim 13, wherein the electrospun textile comprises a mesh.

17. The tissue compression cuff of claim 13, in which the tissue cuff is configured to compress a tissue or otherwise restrict the flow of material or the volume of a biological conduit or pouch.

18. A fistula cuff comprising:
a first fastening portion defining one or more openings;
at least one band comprising:
a first end extending from the first fastening portion at a first end, and
an opposing second end, the at least one band defining a first diameter at the first end and a second diameter at the second end, the first diameter being larger than the second diameter; and
a second fastening portion positioned at the opposing second end of the at least one band, wherein the second end of the at least one band is configured to be inserted through the one or more openings of the first fastening portion,
the tissue compression cuff being sized and configured to reduce blood flow through an arteriovenous fistula and made from an electrospun textile made up of biocompatible nanofibers.

19. The fistula cuff of claim 18, wherein the biocompatible nanofibers comprise a diameter of from 200 nm to about 2000 nm.

20. The fistula cuff of claim 18, wherein the electrospun textile comprises a biologically active agent.
